(19) United States
(12) Patent Application Publication
(10) Pub. No.: US 2019/0128880 A1
(43) Pub. Date: May 2, 2019

(54) METHOD FOR DETECTING THE TARGET IN A SAMPLE

(71) Applicants: Advanced Connection Technology Inc., New Taipei City (TW); National Chung Hsing University, Taichung City (TW)

(72) Inventors: Ching-Chou WU, Taichung City (TW); Ming Jie LIN, Taichung City (TW)

(21) Appl. No.: 15/965,196

(22) Filed: Apr. 27, 2018

(30) Foreign Application Priority Data
Oct. 26, 2017 (TW) 106136945

(51) Int. Cl. G01N 33/543; C12Q 1/26; C12Q 1/54

(52) U.S. Cl. CPC G01N 33/54306; C12Q 1/26; C12Y 101/03004; G01N 33/54326; C12Q 1/54

(57) ABSTRACT

The present invention relates a method for detecting a target in a sample, which can acquire a concentration of the target in a sample by detecting the reaction between a complex and a substrate. The complex comprises a first composition, a target, and a second composition, and the second composition comprises a plurality of enzyme to catalyze the reaction of the substrate.

### TECHNICAL FIELD

The present invention relates to a biomedical detecting method, particularly to a method for detecting a target in a sample.

### BACKGROUND

To enable rapid detection of samples, most of cases are using specific immune response between the analyte and the antibody as the basis for detection. For example, enzyme binding immunosorbent assay, in which the sandwich-method is most commonly used. In simple terms, the principle of enzyme binding immunosorbent assay is to first bind the target in analyte sample to the primary antibody, adding a secondary antibody linked with enzymes after removing the extra primary antibody, after removal of the extra primary antibody, secondary antibody with enzyme is added to bind the target with the secondary antibody containing the enzyme, and after the excess secondary antibody is removed, a substrate that can react with the enzyme is added. By the reaction of enzymes and substrate to achieve quantitative detection purposes.

In the above method, the enzymes are directly labeled on the secondary antibodies. However, the secondary antibody recognition area should avoid being shielded from enzymes, resulting in the limited position of the secondary antibody on which the enzyme can be immobilized, so that the amount of enzyme that can be immobilized on the secondary antibody is extremely small, and if the concentration of the target substance is low, the secondary antibodies that are captured and bound to the labeled enzyme will also decrease, causing the product of enzyme catalytic reaction to drop, resulting in errors in the test results or difficulties in interpretation.

In order to obtain more enzymatic bonds to amplify the signal, at present, the sandwich-type enzyme-binding immunosorbent assay uses magnetic beads that have been labeled with a primary antibody to immunoreact with a test substance, and then use non-magnetic beads with a secondary antibody and enzymes to react immunologically with the analytes to form a sandwich structure, which can be quantified by the catalytic reaction of enzymes. Taking a sucrose substrate catalyzed reaction as an example, a sandwich body labeled with invertase after an immune reaction is dripped in a sucrose solution so that sucrose invertase can convert sucrose to glucose, and after using an existing commercial blood glucose test strip and instrument to detect the catalytic product glucose concentration signal. The concentration of the target substance can be known. Although the non-magnetic beads using fixed primary antibody beads and immobilized enzymes and secondary antibodies can amplify the signal, if the reaction parameters between the invertase and sucrose cannot be accurately controlled, for example, when the mass detection is conducted, the impossibility to accurately control reaction time after titrating sucrose and the inconsistency in the time taken for sampling and instillation of the glucose test strips leads to a difference in the product concentration after the enzyme reaction, resulting in an excessively large error in the measured signal.

### SUMMARY OF THE INVENTION

The main purpose of the present invention is to provide a method for detecting a target in a sample, which can elevate the detection sensitivity and accuracy and avoid the error in individual operation.

Another purpose of the present invention is to provide a method for detecting a target in a sample, which can simplify the procedures of enzyme reaction and the electrochemical measurement to achieve the effect of reducing the cost of detection.

Thus, to accomplish the purpose above, the method disclosed in this present invention can acquire the concentration of the target in a sample by means of the reaction between a complex and a substrate, wherein the complex comprises a first composition, a target, and a second composition, and the second composition comprises a plurality of enzymes for catalyzing the reaction of the substrate

In particularly, the method for detecting a target in a sample disclosed by the present invention comprises the following steps:

Step a, sequentially reacting the sample with the first composition and the second composition, and connecting the first composition and the second composition with the target to obtain the complex.

Step b, the complex being contacted with the substrate to allow the enzymes to catalytically react with the substrate for a predetermined period of time.

Step c, detecting the change of the substrate or the products of catalytic reaction and analyzing the concentration of the target.

The substrate is the material capable of catalytically reacting with the enzyme, for instance, when the enzyme is glucose oxidase, the substrate is glucose.

In order to achieve the efficacy of electrochemical detection of target concentrations, the substrate further comprises an electron transfer substance. For example, the substrate is composed of glucose and potassium ferricyanide. When the substrate is contacted with glucose oxidase, glucose and potassium ferricyanide are catalyzed to obtain potassium ferrocyanide and hydrogen peroxide, therefore, by use of the electrochemical detection method can quickly know the target concentration.

Further, the first composition disclosed in this invention is prepared according to the following steps:

Step a′, taking a plurality of magnetic parts, of which the surfaces comprise a plurality of first binding points;

Step b′, taking a plurality of first connecting parts are used for connecting the target, mixing these first connecting parts and these magnetic connecting parts to locate these first connecting parts on each surface of the magnetic connecting parts by the first binding points.

Wherein, the weight concentration ratio of the first composition and the target is 4.20:1.00, 8.30:1.00, 12.50:1.00 or 6.60:1.00, and preferably is 12.50:1.00.

In an embodiment of the present invention, a glucose oxidase model is used as a ratio of the weight concentration between the first connecting parts and the magnetic parts, and indirectly knowing that when the first connecting parts is an antibody, the first an optimal weight concentration ratio between the connection parts and the magnetic parts.

Specifically, when the magnetic parts consist of the magnetic nanoparticles having a particle diameter of about 100 nm, and the first connecting part is a protein molecule having a molecular weight of 160 kDa, the weight concentration ratio of the first connecting part to the magnetic part is 0.18:1.00-3.70:1.00; or when the first connecting part is a recombinant protein of 52 kDa, such as an anti-adalimumab Fab, the weight concentration ratio of the first connecting part to the magnetic part is 0.13:1.00, 0.26:1.00, 0.65:1.00, 1.30:1.00 or 2.60:1.00.

In order to acquire the first composition with better detecting effect, a step c′ is further provided after step b′, wherein, the step c′ comprises mixing at least one first blockers with these magnetic parts, and connecting the first blockers with the first binding points that is not connected to the first connecting parts.

Specifically, the first binding points in step a′ has an amino group, a carboxyl group or a hydroxyl group, and the first blocker in step c′ is ethanolamine

The second composition disclosed in this invention is prepared by the following steps:

Step a″, taking a plurality of non-magnetic parts of which the surface has a plurality of second binding points;

Step b″, taking a plurality of second connecting parts and a plurality of the enzymes, mixing them in a predetermined ratio to form a mixture, wherein the second connecting parts are used for connecting the target.

Step c″, mixing the mixture of step b″ with these non-magnetic parts, to locate these second connecting parts and the enzymes on the surface of every non-magnetic parts by the second binding points, wherein:

In one example of this invention, glucose oxidase is used as the model to detect the weight concentration ratio between the second connecting parts and the magnetic parts, so that indirectly know the best weight concentration ratio between the second connecting parts and the magnetic parts when the second connecting part is an antibody.

For example, when the non-magnetic part is non-magnetic silica nanoparticle with a particular size of about 100 nm, and the second connecting part is a protein with the molecular weight of about 160 kDa, the weight concentration ratio between the mixture to the non-magnetic parts is between 0.10:1.00 and 2.00:1.00, and the weight concentration ratio between the enzymes and to second connecting parts is between 1.00:1.00 and 9.00:1.00, wherein:

The weight concentration ratio of the mixture to the non-magnetic parts is 0.10:1.00, 0.20:1.00, 0.50:1.00, 1.00:1.00 or 2.00:1.00, and the best weight concentration ratio between the non-magnetic parts and the mixture is preferably 2:1; the weight concentration ratio of the enzymes to the second connecting parts is equal to 1.00:1.00, 3.00:1.00 or 9.00:1.00, and the best weight concentration ratio between the enzymes and the second connecting parts is preferably 3.00:1.00.

In order to acquire the second composition with a better detecting effects, a step d″ is provided after step c″, wherein the step d″ is to take at least one second blocker to mix with these non-magnetic parts, and connect the second blocker to the second binding points that is not connected to the second connecting parts.

Specially, the second binding points of step a″ comprise an amine group, a carboxyl group or a hydroxyl group, and the second blocker in step d″ is ethanolamine

### BRIEF DESCRIPTION OF THE DRAWINGS

FIG. 1 is the process for preparing the first composition of the present invention.

FIG. 2 is the process for preparing the second composition of the present invention.

FIG. 3 is the cyclic voltammogram obtained by cyclic voltammetry with electrodes used in the examples of this present invention.

FIG. 4A is the amperommogram (+0.2 V vs. carbon) for the magnetic beads after being modified by the first connecting parts (GOx) with different concentrations, respectively dissolved in the glucose and potassium ferricyanide solution and dropped on a two-electrode typed screen printing carbon electrode, in which, the blank group is thick solid line, in which the weight percentage of glucose oxidase to magnetic beads is 0.18:1.00, 0.37:1.00, 0.92:1.00, 1.84:1.00 and 3.70:1.00 represented by thick dashed line, dashed line, solid line, short dashed line and point chain line, respectively.

FIG. 4B is the chart showing the current increment (ΔI=I_{Gox:MB}-I_{blank}) of the samples for FIG. 4A at the 2.5 second (dashed line), 5.0 second (solid line) and 10.0 second (short dashed line) respectively, wherein MB represents the magnetic beads, and GOx represents glucose oxidase.

FIG. 4C is the amperommogram (+0.2 V vs. carbon) for the magnetic beads after being modified by the first connecting parts (recombinant protein, Fab) with different concentrations, respectively immunoreact with adalimumab and the second connecting parts (GOx-labeled secondary antibody), then dissolved in the glucose and potassium ferricyanide solution and dropped on a two-electrode typed screen printing carbon electrode, in which, the blank group is thick solid line, in which the weight percentage of the first connecting parts to the magnetic beads is 0.13:1.00, 0.26:1.00, 0.65:1.00, 1.30:1.00 and 2.60:1.00 represented by thick dashed line, dashed line, solid line, short dashed line and point chain line, respectively.

FIG. 4D is the chart showing the current increment (ΔI=I_{Fab:MB}-I_{blank})of the samples for FIG. 4C at the 2.5 second (dashed line), 5.0 second (solid line) and 10.0 second (short dashed line) respectively, wherein MB represents the magnetic beads, and Fab represents recombinant protein.

FIG. 5A is the amperommogram (+0.2 V vs. carbon) after modifying the non-magnetic beads using glucose oxidase at different concentrations, catalytically reacting with glucose and potassium ferricyanide, respectively. The blank group is solid line, and the weight percentage of glucose oxidase to the non-magnetic beads is 0.10:1.00, 0.20:1.00, 0.50:1.00, 1.00:1.00 and 2.00:1.00 represented by the thick dashed line, dashed line, solid line, short dashed line and point chain line, respectively.

FIG. 5B is the chart showing the current increment (ΔI=I_{GOx:NMB}-I_{blank}) of samples for FIG. 5A at 2.5 second (dashed line), 5.0 second (solid line) and 10.0 second (short dashed line), respectively, wherein, NMB represents the bead, and GOx represents glucose oxidase.

FIG. 6 is the amperommogram (+0.4 V vs. Ag/AgCl) obtained in performing the electrochemical analysis by means that the glucose oxidase in different concentration ratios and the second connecting parts modified non-magnetic beads are respectively reacted with the modified adalimumab/MPA/Au electrode, and the electrode is placed in glucose and potassium ferricyanide solution to perform immune reaction. The weight concentration percentage (ratio) of glucose oxidase to the second connecting parts are 1.00:1.00, 3.00:1.00 and 9.00:1.00 represented by thick solid line, solid line, short dashed line, respectively, where the inner panel presents the current increment (ΔI=I_{40 s}-I_{20 s}) by deducting background values of sample. GOx represents glucose oxidase, and 2^{nd} Ab represents the second connecting parts.

FIG. 7 is the amperommogram (+0.2 V vs. carbon) obtained when the first composition (the magnetic bead modified by adalimumab) reacts with the second composition (non-magnetic beads modified by glucose oxidase and secondary antibodies) at different concentrations, and then catalytically reacts respectively with glucose and potassium ferricyanide solution. The blank group is thick solid line, the groups that the weight concentrations percentage of the second composition to the first composition is 9.20:1.00, 18.4:1.00 and 36.80:1.0 represented by short dashed line, solid line and dashed line, respectively. While the inner panel presents the current increment (ΔI=I_{SC:FC}-I_{blank}) of sample in this main figure, where dashed line, solid line and short dashed line represent sampling at the 2.5 second, 5 second and 10 second, respectively. SC represents the second composition, and FC represents the first composition.

FIG. 8 is the amperommogram (+0.2 V vs. carbon) when the first composition immunoreacts with the saturated amount of second antibody labeled by glucose oxidase after immune reacting with adalimumab (Ada) in different concentration, to form complexes, each of the complexes catalytically reacts with glucose and potassium ferricyanide; wherein the blank group is thick solid line , the group that the weight percentage of the first composition to target is 4.20:1.00, 8.30:1.00, 12.50:1.00 and 16.60:1.00 represented by short dashed line, solid line, dashed line and thick dashed line, respectively, while the inner panel represents the current increment (ΔI=I_{FC:Ada}-I_{blank}) of sample in this main figure, and dashed line, solid line and short dashed line represent the sampling at 2.5 second, 5 second and 10 second, respectively. FC represents the first composition, Ada represents adalimumab, and FC:Ada represents the first composition immunoreacted by adalimumab.

FIG. 9 is the result obtained in electrochemical analysis after reacting the adalimumab target in different concentrations and the first composition and then the second composition to form the complex, wherein, the group that the concentration of adalimumab target is 0.00 μg/mL, 0.10 μg/mL, 0.50 μg/mL and 1.00 μg/mL represented by thick solid line, short dashed line, solid line and dashed line, respectively, while the inner panel represents the current increment (ΔI=I_{Ada}-I_{blank}) of sample in this main figure, the dashed line, solid line and short dashed line represent sampling at 2.5 second, 5 second and 10 second, respectively.

### DETAILED DESCRIPTION OF THE EMBODIMENTS

The present invention discloses a method for detecting a target in a sample, which accomplishes the detection by firstly reacting the complex and the substrate, then detecting the change of the substrate or the change of reaction product. And the method for detecting the change of the substrate or the change of reaction product after the reaction is a well-known technique in the field of the present invention, such as an electrochemical method, an enzyme coloring method, a fluorescent cursor method, and the like.

Specifically, the complex comprises a first composition, a target, and a second composition, and the second composition comprises an enzyme which is catalytically reacted with the substrate. For example, when the sample comprises a target, after the sample is sequentially reacted by the first composition and the second composition, the target is connected to the first composition and the second composition to form a complex.

In order to help the promotion and use of the method of detecting the target of the present invention in the sample, it can react with commercially available electrodes. Generally, commercially available electrodes are coated on a substrate material with a substrate and an electron transfer substance, wherein:

The substrate material is made of a conductive material, such as the screen-printed carbon electrode, Iridium tin oxide, carbon, graphite, gold or platinum.

The substrate corresponds to the enzymes in the complex, for example when the enzyme is glucose oxidase, the substrate is glucose; when the enzyme is horseradish peroxidase, the substrate is hydrogen peroxide, and when the enzyme is alkaline phosphatase, the substrate is 4-Methylumbelliferyl phosphate or p-Nitrophenyl and when enzyme is invertase, the substrate is sucrose.

The electron transfer substance may be ferricyanide, ferrocene, hydroquinone, thionine, methylene blue, 1,1-dicarboxylic acid ferrocene or Ru (bpy)3^{3+/2+}. For example, when the commercial available electrode is modified by glucose and potassium ferricyanide, the catalytical reaction of the complex disclosed by the present invention is catalyzed by the glucose oxidase on the second composition, which can catalyze glucose and potassium ferricyanide into potassium ferrocyanide and hydrogen peroxide, while the concentration of sample can be obtained by detecting the total oxidation current changes before and after the reaction before and after the reaction through the amperometry, coulometry or voltammetry method.

Furthermore, the scientific terms not defined in the specification of the present invention may be interpreted according to the general knowledge in the technical field to which the invention pertains.

The magnet defined in this invention is the permanent magnet or electromagnet. The term purification defined in this present invention means purifying using size limiting method or magnetic adsorption method, wherein the size limiting method is a method of obtaining a separation material by using a size change before and after the bonding and a tool such as a filter membrane; and a magnetic adsorption method means separating the magnetic material and the non-magnetic material through absorbing the magnetic material by the magnetic force.

The first binding points or the second binding points disclosed in this present invention means a functional groups able to be chemically activated, and the groups can be covalently bonded to proteins by chemical modification, such as amine group, carboxyl group or hydroxyl group. In the present invention is concerned, the first binding points or the second binding points are provided on the magnetic or non-magnetic surface based on the surface modification technology.

In the following several examples and the figures, present invention will be further described.

### EXAMPLE 1

### Preparation of the First Composition

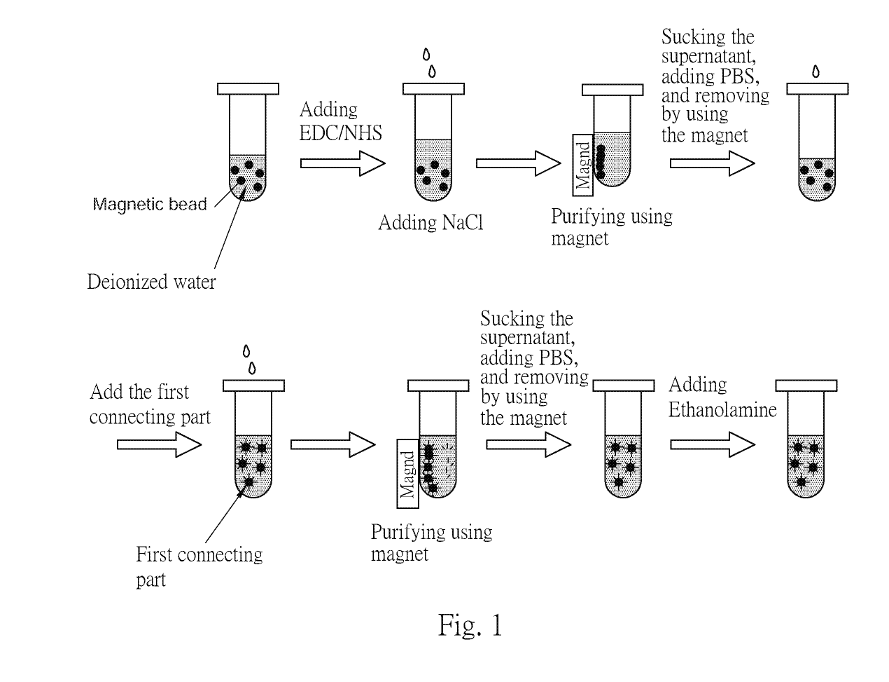

First, referring to the FIG. 1, took the magnetic bead solution (prepared in deionized water), the surfaces of every magnetic beads were modified by the functional groups of COOH, and the EDC/NHS mixture solution (in MES of 100 mM, pH 4.6), mixed with the same volume ratio, after standing for 30 minutes, added the sodium chloride solution at a concentration of 50 mM or more, adsorbed the magnet beads using magnet, after removing the supernatant added the phosphate buffer at a concentration of 10 mM to detach the beads, and obtain 100 nm beads/PBS solution.

The first connecting parts solution was dropped into the magnetic beads/PBS solution, uniformly mixed and allowed to stand. Then, sodium chloride having a concentration of 50 mM or more was added, and the magnetic beads were adsorbed by a magnet to remove the supernatant and the unbound first connecting parts. The magnetic beads were purified, phosphate buffer was added, and then 50 μL of ethanolamine (prepared in deionized water) was added to fill the unbound activated COOH functional groups on the surface of the magnetic beads. After completion, the first composition was adsorbed and purified with a magnet.

### EXAMPLE 2

### Preparation of the Second Composition

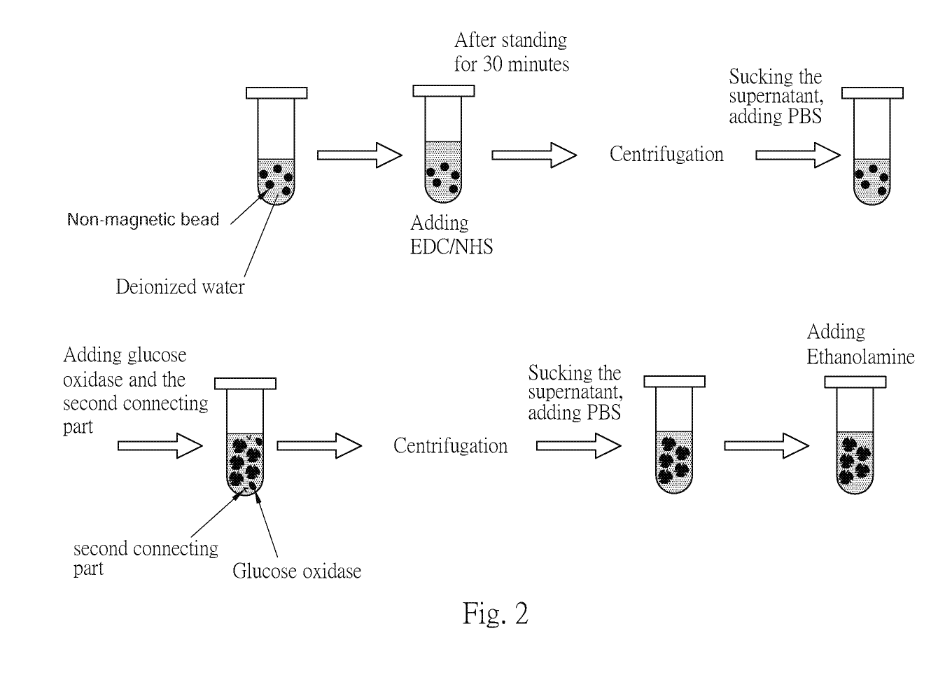

Referring to the figure FIG. 2, the non-magnetic beads were taken, the surface of which was modified with COOH functional groups. The non-magnetic beads were mixed with EDC/NHS in the same volume, allowed to stand for more than 30 minutes, and then remove residual EDC/NHS in the solution by centrifugation, wherein the centrifugal speed is preferably 6000 rpm or more. 100 nm of the collected activated non-magnetic bead solution was added to a mixture of enzyme (50 μL) and a second connecting parts (50 μL), and after mixing evenly for a predetermined period of time, the non-magnetic beads were purified by a centrifuge. The supernatant and the unbound enzyme and the second connecting parts were removed, and 50 μL of ethanolamine (deionized water) was added to fill the unbound COOH functional groups on the surface of the non-magnetic beads, and finally centrifuged to remove the unbound ethanolamine to obtain the second composition.

### EXAMPLE 3

### Analysis on the Composition Ratio of the First Composition and the Second Composition

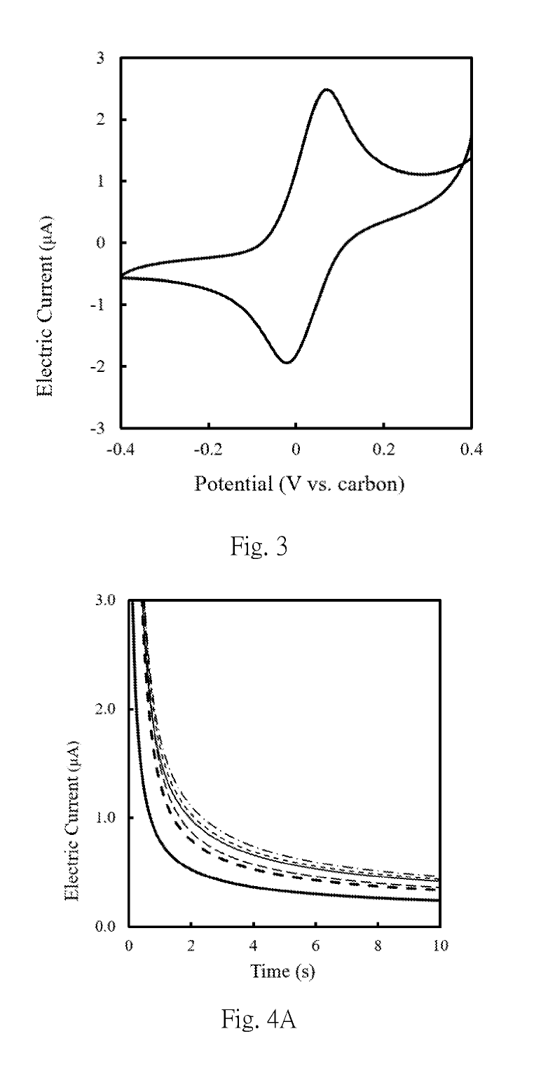

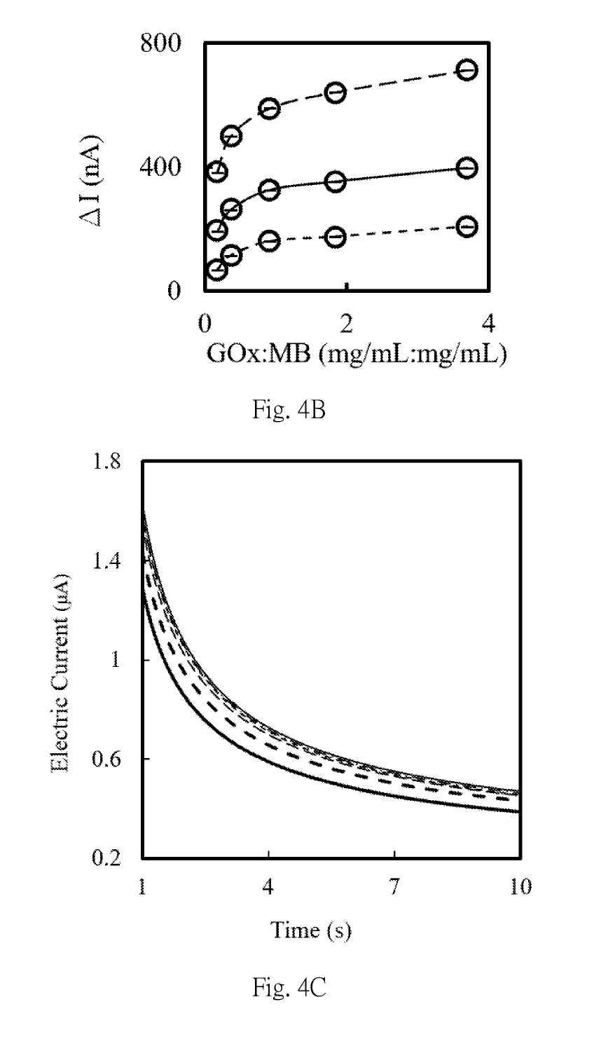

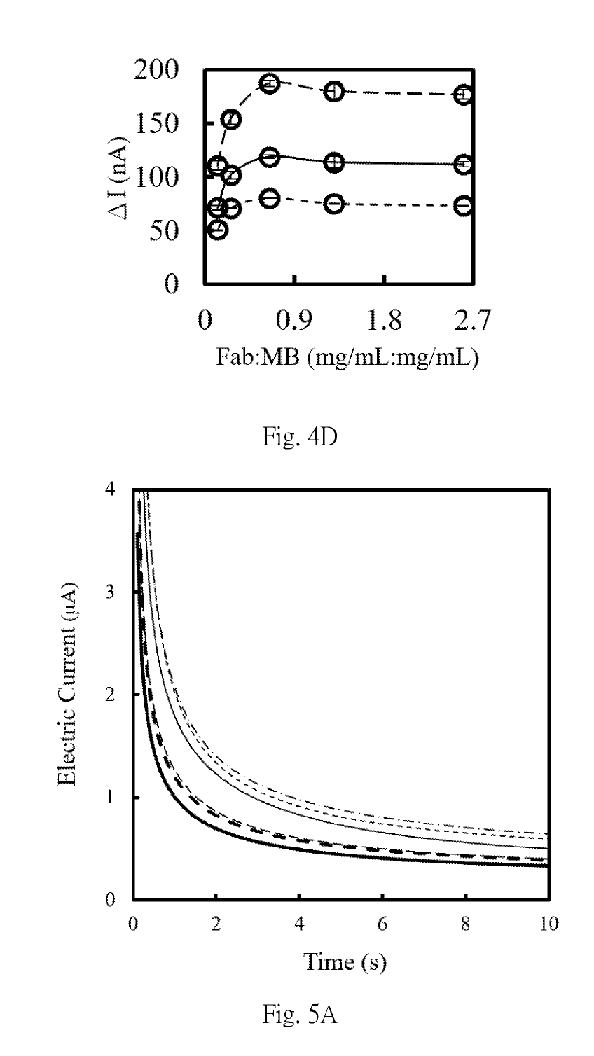

A 5 μl 10 mM phosphate buffer containing 200 mM glucose and 200 mM potassium ferricyanide was applied to a two-electrode typed screen printing carbon electrode (hereinafter abbreviated as SPCE electrode), and the results of the cyclic amperometric assay were shown in FIG. 3, the detection potential suitable for the amperometric measurement is set to be +0.05 V to +0.5 V, and the detection time is 1 to 100 seconds. The first composition was prepared according to the method shown in Example 1, wherein the first connecting part was glucose oxidase, which was a protein having a molecular weight of about 160 kDa, and mixed the weight of the first connecting parts and the magnetic beads by concentration ratios (mg/mL: mg/mL) of 0.18:1, 0.37:1, 0.92:1, 1.84:1 or 3.70:1, respectively, to prepare first composition solutions obtained by mixing at different weight concentration ratios. 5 μL each of the first composition solution was mixed with 5 μL of a 10 mM phosphate buffer containing 200 mM glucose/potassium ferricyanide and then dropped on the SPCE electrode, and the results of the electrochemical detection were as shown in FIG. 4A and 4B. The applied voltage was +0.2 V (vs. carbon).

Furthermore, the first composition was prepared according to the method shown in Example 1, wherein the first connecting part was anti-adalimumab Fab protein and the weight concentration ratio of the first connecting part and magnetic beads was 0.13:1.00, 0.26:1.00, 0.65:1.00, 1.30:1.00 or 2.60:1.00. The second composition was prepared according to the method described before, wherein the second connecting parts was goat anti-human antibody labeled by glucose oxidase. And the each first composition was sequentially immunoreacted with 5 μL adalimumab and 10 μL of the second composition, then to mixed with 10 mM phosphate buffer containing 200 mM glucose/potassium ferricyanide, wherein 5 μL of each was used to mix, and after mixing, dropped on the SPCE electrode. The result of the electrochemical analysis was shown in FIG. 4C and FIG. 4D, where the applied voltage was +0.2V (vs. carbon). FIG. 4C and FIG. 4D shows that when the ratio of the weight concentration of the first connecting part to the magnetic bead solution (mg/mL: mg/mL) is 0.65:1:00, the saturation has been reached.

From the results of FIG. 4, it can be seen that when the weight concentration ratio of glucose oxidase and magnetic beads is 0.92:1.00, and the weight concentration ratio of Fab and magnetic beads is 0.65:1.00, the saturation tends to occur, and those skilled in the art and having ordinary knowledge can deduct from the above results that when the first connecting part is set to a recombinant protein having a molecular weight of 52 kDa, the weight concentration ratio of the first connecting part to the magnetic beads (mg/mL: mg/mL) is 0.13:1.00, 0.26:1.00, 0.65:1.00, 1.30:1.00 or 2.60:1.00.

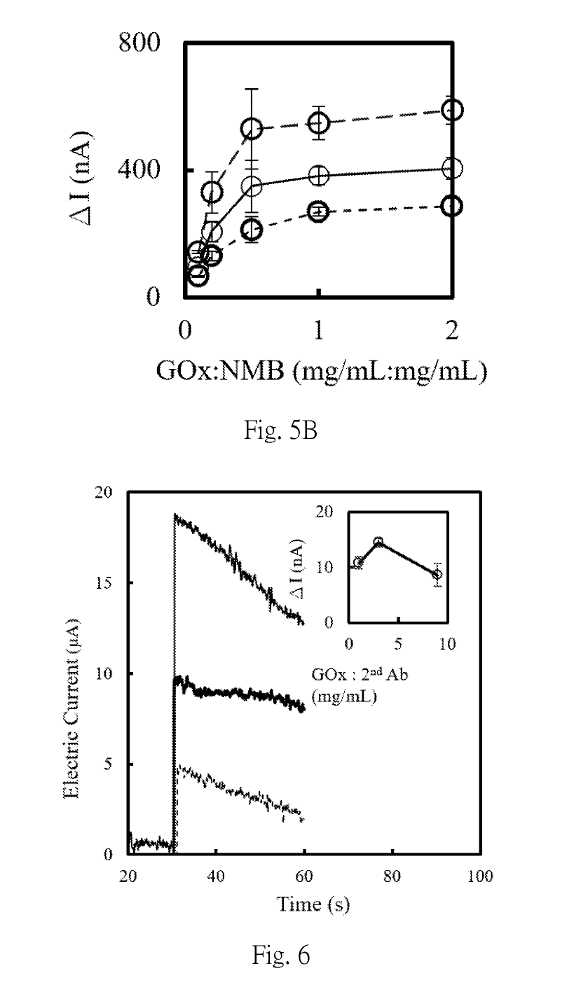

According to the method shown in Example 2, the modified material is the non-magnetic beads made of silicon oxide, and non-magnetic beads solution was taken and modified with different concentrations of glucose oxidase, wherein glucose oxidase and activated the non-magnetic bead solution has a weight concentration ratio (mg/mL: mg/mL) of 0.00:1.00, 0.10:1.00, 0.20:1.00, 0.50:1.00, 1.00:1.00 or 2.00:1.00, and then each of the modified non-magnetic beads were mixed with 10 mM phosphate buffer containing 200 mM glucose/potassium ferricyanide and 5 μL of each was used to mix, and then dropped on the SPCE electrode. The result of the electrochemical analysis was shown in FIG. 5, where the applied voltage was +0.2V (vs. carbon). From the results in FIG. 5, it can be seen that when the ratio of the weight concentration of glucose oxidase to the activated non-magnetic bead solution (mg/mL: mg/mL) is 0.5:1:00, the saturation has been reached.

In addition, the glucose oxidase and the second connecting parts are first mixed in the following concentration ratios (mg/mL: mg/mL): 1.00:1.00, 3.00:1.00 or 9.00:1.00, respectively, and then the non-magnetic beads are modified with the mixed glucose oxidase/secondary connecting parts solution respectively to obtain the second composition, wherein the second connecting parts was goat anti-human antibody (160 kDa) and the enzyme was glucose oxidase. After each of the second compositions was immunologically reacted with an electrode modified by adalimumab/MPA/Au, in 200 mM potassium ferricyanide 10 mM phosphate buffer (30 seconds prior to) and in 200 mM glucose/potassium ferricyanide, electrochemical analysis (+0.45 V vs. Ag/AgCl) was performed in 10 mM phosphate buffer (after 30 seconds). The results are shown in FIG. 6. As is clear from the results of FIG. 6, in the second composition disclosed in the present invention, the optimal weight concentration ratio between the glucose oxidase and the second connecting parts was 3.00:1.00.

### EXAMPLE 4

### Analyzing the Ratio of Magnetic Beads to Non-Magnetic Bead

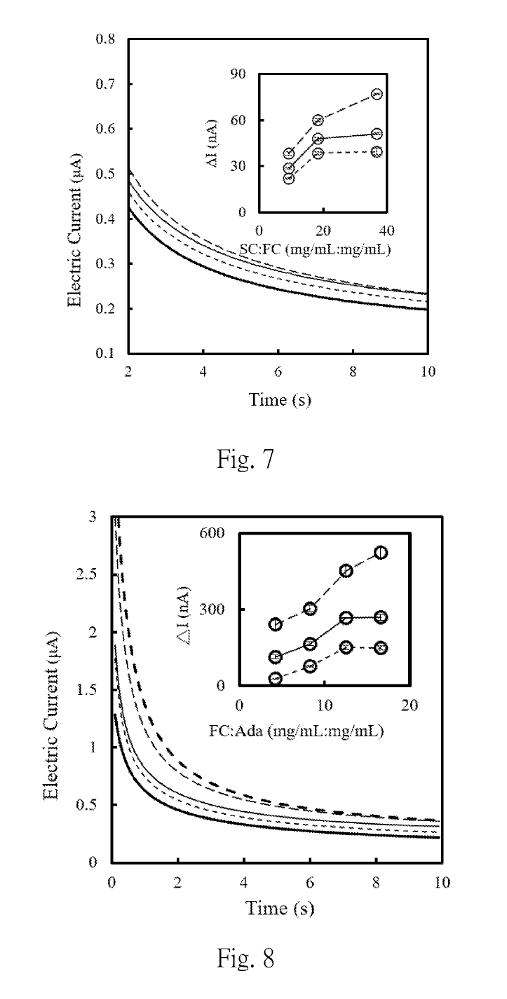

The surface of the magnetic beads was activated with reference to Example 1, and the magnetic beads were modified with adalimumab, and the second composition was prepared with reference to the method shown in Example 2, wherein the second connecting parts was goat anti-human antibody and the enzyme was glucose oxidase. The second composition and the adalimumab-modified magnetic beads were subjected to a proportional reaction at the following weight concentration ratios (mg/mL: mg/mL): 0.00:1.00, 9.20:1.00, 18.40:1.00 or 36.80:1.00, respectively, and purified to obtain each complex. 5 μL of each of the complexes was taken to mix with 200 mM glucose/potassium ferricyanide in 10 mM phosphate buffer (5 μL), and was dropped onto a SPCE electrode for electrochemical analysis. The voltages used were +0.2V (vs. carbon). The result is shown in the FIG. 7. The result from FIG. 7 shows that the ratio of the second composition (called SC) (non-magnetic beads modified by the antibody and the glucose oxidase) to the first composition (called FC) (magnetic beads modified by adalimumab) has become saturated at 18.40:1.00 (mg/mL: mg/mL). The non-magnetic beads and the magnetic beads were calculated based on the unmodified weight.

### EXAMPLE 5

### Analysis of the Ratio of the First Composition to the Target

A first composition is prepared according to the method shown in Example 1, wherein the first connecting part was an anti-adalimumab Fab protein having a molecular weight of about 52 kDa.

The first composition is immunologically reacted with different concentrations of adalimumab to obtain the adalimumab modified first composition, and after purification, each of the adalimumab modified first compositions is further immunologically reacted with a saturated amount of a goat anti-human antibody labeled with a glucose oxidase to form a complex, wherein the weight concentration ratio (mg/mL: mg/mL) of the first composition to adalimumab is 0.00:1.00, 4.20:1.00, 8.30:1.00, 12.50:1.00 or 16.60:1.00. 5 μL of each purified complex was mixed respectively with 200 mM glucose/potassium ferricyanide in 10 mM phosphate buffer (5 μL). The result of electrochemical analysis was shown in FIG. 8.

From the result of FIG. 8, it is shown that the weight ratio of the first composition to adalimumab has become saturated at 12.50:1.00, wherein the weight of the first composition is calculated only when the beads are unmodified.

### EXAMPLE 6

### Detection of Adalimumab

The first composition and the second composition are prepared by the methods shown in Examples 1, 2 and 3, wherein the first connecting part is an anti-adalimumab Fab recombinant protein (anti adalimumab Fab) and the second connecting part is a goat anti-human antibodies.

A solution of adalimumab (in phosphate buffer) at a concentration of 0.00, 0.10 μg/mL, 0.50 μg/mL or 1.00 μg/mL was immunologically reacted with the first composition to capture adalimumab by the first connecting part. In order to link the first composition with adalimumab, the first composition is then adsorbed with a magnet to remove adalimumab that is unbound to the first composition. A second predetermined amount of the second composition is then added to allow the second connecting part to immunoreact with adalimumab. The first composition, adalimumab, and the second composition can form a complex by performing an immunoreaction with the adalimumab antibody on the second composition, wherein the predetermined amount is calculated based on the ratio of nonmagnetic beads to magnetic beads in Example 4 is 18.40:1.00 (mg/mL: mg/mL). Finally, the composite is adsorbed by a magnet to remove the second composition that is unbound.

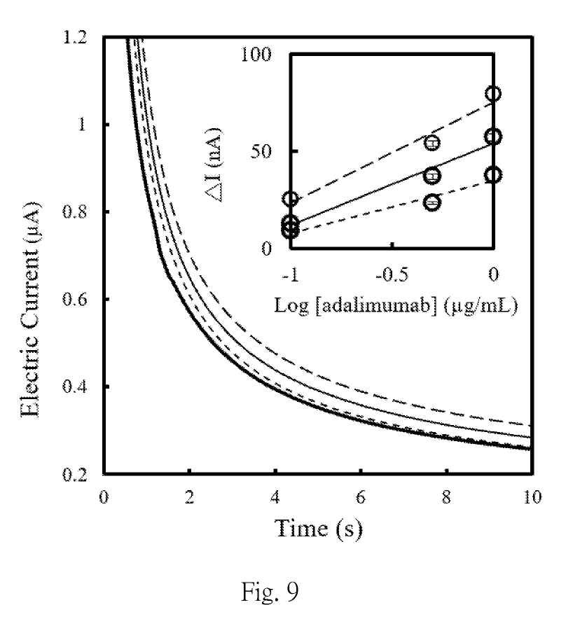

5 μL of each complex formed by reacting with different concentrations of adalimumab and 5 μL of 10 mM phosphate buffer containing 200 mM glucose/potassium ferricyanide was mixed, and then dropped onto the SPCE electrode to electrochemically analyze. The results are shown in FIG. 9, where the voltage used was +0.2 V (vs. carbon). It is shown in FIG. 9 that the method disclosed in the present invention can use the first composition and the second composition, respectively, to sequentially connect with the target to form a complex, and by detecting the enzyme-catalyzed reaction on the complex, the current of the product can be changed to obtain the calibration curve of the target.

The invention claimed is:

1. A method for detecting a target in a sample, acquire the concentration of the target in a sample by detecting the reaction between a complex and a substrate, wherein characterized in that:
the complex has a first composition, a target and a second composition; and
the second composition has a plurality of enzymes for catalyzing the reaction of the substrate.

2. The method for detecting a target in a sample according to claim 1, wherein the method comprises the following steps:
a) the sample sequentially reacting with the first composition and the second composition to allow the first composition and the second composition to be connected with the target to obtain the complex;
b) the complex contacting with the substrate to allow the plurality of enzymes catalytically react with the substrate for a predetermined period of time; and
c) detecting the change of the substrate or the products of catalytic reaction for analyzing to obtain the target's concentration.

3. The method for detecting the target in a sample according to claim 2, wherein the substrate is glucose, and the enzyme is glucose oxidase.

4. The method for detecting the target in a sample according to claim 2, wherein the substrate consists of glucose and electron transfer substance, and is disposed on a wafer, and the enzyme is glucose oxidase.

5. The method for detecting the target in a sample according to claim 2, wherein the first composition is prepared by the following steps:
a′) taking a plurality of magnetic parts of which the surfaces have a plurality of first binding points; and.
b′) taking a plurality of first connecting parts used for reacting with the target, mixing the first connecting parts and the magnetic connecting parts to locate the the first connecting parts on each surface of the magnetic parts by the first binding points.

6. The method for detecting a target in a sample according to claim 5, wherein the weight concentration ratio of the first composition to the target is selected from a group consisting of the following ratios of 4.20:1.00, 8.30:1.00, 12.50:1.00 and 16.60:1.00.

7. The method for detecting a target in a sample according to claim 5, wherein when the first connecting part is a protein with a molecular weight of about 160 kDa, the weight concentration ratio of the first connecting part to the magnetic part is between 0.18:1.00 and 3.70:1.00.

8. The method for detecting a target in a sample according to claim 5, wherein when the first connecting part is a protein with a molecular weight of about 52 kDa, the weight concentration ratio of the first connecting part to the magnetic part is selected from the group consisting of the following ratios of 0.13:1.00, 0.26:1.00, 0.65: 1:00, 1.30: 1.00 and 2.60: 1.00

9. The method for detecting a target in a sample according to claim 5, wherein the method further comprises a step c′ provided after the step b′, in the step c′ at least one first blocker is used to mix with the plurality of magnetic parts and connect the first blocker to the first binding points not connected to the first connecting parts.

10. The method for detecting a target in a sample according to claim 5, wherein the first binding points of the step a′ comprise an amine group, a carboxyl group or a hydroxyl group, and the first blockers in the step c′ are ethanolamine

11. The method for detecting a target in a sample according to claim 2, wherein the second composition is prepared by the following steps:
a″) taking a plurality of non-magnetic parts of which the surfaces have a plurality of second binding points;
b″) taking a plurality of second connecting parts and a plurality of the enzymes to form a mixture, the second connecting parts are used for connecting the target;
c″) mixing the mixture of step b″ with the non-magnetic parts to locate the second connecting parts and the enzymes on each surface of the non-magnetic parts by the second binding points.

12. The method for detecting a target in a sample according to claim 11, wherein when the second connecting part is a protein with a molecular weight of about 160 kDa, the weight concentration ratio of the mixture to the non-magnetic is between 0.10: 1.00 and 2.00:1.00, and the weight concentration ratio of the enzymes to the second connecting parts is between 1.00:1.00 and 9.00:1.00.

13. The method for detecting a target in a sample according to claim 12, wherein the weight concentration ratio of the mixture to the non-magnetic parts is selected from the group consisting of the following ratios of 0.10:1.00, 0.20:1.00, 0.50:1.00, 1.00:1.00 and 2.00:1.00.

14. The method for detecting a target in a sample according to claim 13, wherein the weight concentration ratio of the non-magnetic parts to the mixture is 2:1.

15. The method for detecting a target in a sample according to claim 12, wherein the weight concentration ratio of the enzymes to the connecting parts is selected from the group consisting of the following ratios of 1.00:1.00, 3.00:1.00 and 9.00:1.00.

16. The method for detecting a target in a sample according to claim 15, wherein the weight concentration ratio of the enzymes to the second connecting parts is 3.00:1.00.

17. The method for detecting a target in a sample according to claim 11, wherein the method further comprises a step d″ provided after the step c″, in the step d″ at least one second blocker is used to mix with the non-magnetic parts to connect the second blocker with the second binding points not connected with the second connecting parts.

18. The method in a sample according to claim 17, wherein the second binding points in step a″ comprise an amine group, a carboxyl group or a hydroxyl group, and the second blockers in step d″ are ethanolamine
